(19) United States
(12) Patent Application Publication
(10) Pub. No.: US 2025/0018164 A1
(43) Pub. Date: Jan. 16, 2025

(54) LIQUID MEDICINE ADMINISTRATION DEVICE

(71) Applicant: TERUMO KABUSHIKI KAISHA, Tokyo (JP)

(72) Inventor: Kentaro NAKAJIMA, Nakakoma-gun (JP)

(73) Assignee: TERUMO KABUSHIKI KAISHA, Tokyo (JP)

(21) Appl. No.: 18/900,311

(22) Filed: Sep. 27, 2024

Related U.S. Application Data
(63) Continuation of application No. PCT/JP2023/004286, filed on Feb. 9, 2023.

(30) Foreign Application Priority Data
Mar. 30, 2022 (JP) 2022-056882

(51) Int. Cl. A61M 39/12; A61M 5/14; A61M 5/142; A61M 39/10

(52) U.S. Cl. CPC A61M 39/12; A61M 5/1413; A61M 5/142; A61M 2005/1402; A61M 2039/1027

(57) ABSTRACT

A liquid medicine administration device includes: a catheter configured to be inserted into and left in place in a living body; a tube configured to allow a liquid medicine to flow toward the catheter; a connector located at a proximal portion of the tube, the connector being selectively connectable to (i) a priming solution delivery tool configured to deliver a priming solution from a distal portion of the priming solution delivery tool, and (ii) a prefilled syringe filled with the liquid medicine; and a hollow needle located inside the connector and protruding in a proximal direction. The connector includes: a first connection portion connectable to the prefilled syringe, and a second connection portion located at a position different from the first connection portion and connectable to the priming solution delivery tool.

### CROSS-REFERENCE TO RELATED APPLICATIONS

This is a bypass continuation of PCT Application No. PCT/JP2023/004286, filed on Feb. 9, 2023, which claims priority to Japanese Patent Application No. 2022-056882, filed on Mar. 30, 2022. The entire contents of these application are incorporated herein by reference.

### BACKGROUND

The present disclosure relates to a liquid medicine administration device having a connector selectively connectable to a priming solution delivery tool and a prefilled syringe.

The applicant of the present application has proposed a liquid medicine administration device for administering a desired dose of a liquid medicine to a living body at an appropriate timing (see WO 2018/173962 A). The liquid medicine administration device is connected with a patch-type tube with needle. The patch-type tube with needle includes a connector connectable to a nozzle portion of a cylindrical body filled with a liquid medicine, and the connector is connected to a puncture needle protruding from a patch portion via a liquid delivery tube. After the connector is connected to the nozzle portion of the cylindrical body and the puncture needle punctures the skin of a patient, the liquid medicine within the cylindrical body is administered into the body of the patient through the tube with needle.

### SUMMARY

A liquid medicine administration device administers a liquid medicine into the body of a patient through a catheter that is left in place under the skin of the patient, and it is desirable to clean a liquid delivery tube by supplying a priming solution into the liquid delivery tube before the administration of the liquid medicine, and then, to connect a cylindrical body filled with the liquid medicine to a connector to administer the liquid medicine.

(1) According to one aspect of the present disclosure, a liquid medicine administration device includes: a catheter that is to be inserted into and left in place in a living body; a tube capable of allowing a liquid medicine to flow toward the catheter; a connector provided at a proximal portion of the tube, the connector being selectively connectable to a priming solution delivery tool capable of delivering a priming solution charged inside from a distal portion and a prefilled syringe filled with the liquid medicine; and a hollow needle provided inside the connector and protruding in a proximal direction, wherein the connector includes a first connection portion connectable to the prefilled syringe, and a second connection portion provided at a position different from the first connection portion and connectable to the priming solution delivery tool.

According to the liquid medicine administration device, the connector can be connected to the priming solution delivery tool via the second connection portion, whereby priming can be performed before the prefilled syringe of a device body is connected. When the prefilled syringe is connected to the first connection portion of the connector, pressure in the distal direction is not applied by the prefilled syringe, so that leakage of the liquid medicine in the distal direction is prevented.

(2) In the liquid medicine administration device according to (1), the first connection portion may be provided in a first connection member connected to the proximal portion of the tube, and the second connection portion may be provided in a second connection member detachably attached to a proximal portion of the first connection member.

(3) In the liquid medicine administration device according to (2), the second connection member may include a first cylindrical body that has a cylindrical shape and that is connected to a distal portion of the priming solution delivery tool, and a second cylindrical body that covers an outside of the first cylindrical body and is engaged with the first connection member.

(4) In the liquid medicine administration device according to (3), the second cylindrical body may include an arm that is disposed outside the first connection portion of the first connection member and is tiltable in a direction approaching or away from the first connection portion, and the arm may include an engagement portion that engages with an engagement hole formed in an outer peripheral surface of the first connection portion.

(5) In the liquid medicine administration device according to any one of (1) to (4), the first connection portion and the second connection portion may be different regions of a single connection member.

(6) In the liquid medicine administration device according to (5), the hollow needle may be integrally molded with the connection member.

(7) In the liquid medicine administration device according to any one of (1) to (6), the second connection portion may have a fitting hole into which a distal nozzle portion of the priming solution delivery tool is inserted and engageable, the fitting hole having a tapered shape and facing a distal end of the hollow needle.

(8) The liquid medicine administration device according to (3) or (4) may include a cap held by at least one of the first cylindrical body or the second cylindrical body and punctured with the hollow needle.

According to certain embodiments of the present disclosure, the connector can be connected to the priming solution delivery tool via the second connection portion, whereby priming can be performed before the prefilled syringe of the device body is connected. When the prefilled syringe is connected to the first connection portion of the connector, pressure in the distal direction is not applied by the prefilled syringe, so that leakage of the liquid medicine in the distal direction is prevented.

### BRIEF DESCRIPTION OF THE DRAWINGS

FIG. 1 is an overall plan view of a liquid medicine administration device according to a first embodiment.

FIG. 2 is an overall cross-sectional view of the liquid medicine administration device illustrated in FIG. 1.

FIG. 3 is an overall plan view illustrating a state in which a device body is attached to a connector of the liquid medicine administration device illustrated in FIG. 1.

FIG. 4 is an overall cross-sectional view illustrating a state in which a priming solution delivery tool is attached to the connector of the liquid medicine administration device illustrated in FIG. 2.

FIG. 5 is an enlarged cross-sectional view illustrating the vicinity of a needle portion in the liquid medicine administration device illustrated in FIG. 2.

FIG. 6 is an enlarged plan view illustrating the vicinity of a distal end of the liquid medicine administration device illustrated in FIG. 1.

FIG. 7 is an enlarged plan view illustrating the vicinity of a distal end of a liquid medicine administration device having a holder according to a modification.

FIG. 8 is an enlarged plan view of the vicinity of the distal end in FIG. 6 as viewed from a back surface.

FIG. 9A is a cross-sectional view taken along line IXA-IXA in FIG. 1, and FIG. 9B is an enlarged cross-sectional view of a catheter holding portion in FIG. 9A.

FIG. 10 is an enlarged side view of the vicinity of the needle portion in FIG. 6 as viewed from the side.

FIG. 11 is a plan view of the holder as viewed from a base portion side.

FIG. 12 is an enlarged cross-sectional view illustrating the vicinity of the connector in the liquid medicine administration device illustrated in FIG. 2.

FIG. 13 is an enlarged cross-sectional view of the vicinity of a valve body in the liquid medicine administration device illustrated in FIG. 12.

FIG. 14 is a front view of the valve body illustrated in FIG. 13 as viewed in a distal direction.

FIG. 15 is an enlarged cross-sectional view illustrating the vicinity of a connection portion with the prefilled syringe in the liquid medicine administration device illustrated in FIG. 2.

FIG. 16 is an enlarged cross-sectional view illustrating the vicinity of a connector in a liquid medicine administration device according to a second embodiment.

FIG. 17 is an enlarged cross-sectional view illustrating a state in which a priming solution delivery tool is attached to the connector illustrated in FIG. 16.

FIG. 18 is an enlarged cross-sectional view illustrating a state in which a device body is attached to the connector illustrated in FIG. 16.

### DETAILED DESCRIPTION

A liquid medicine administration device 10 according to a first embodiment is configured to be left in place on a body surface 12a of, for example, the abdomen of a user (living body) 12 and to automatically administer a liquid medicine S1 into the body. For example, the liquid medicine administration device 10 is used to administer the liquid medicine S1 to the user 12 at a timing when a predetermined time elapses after a medical treatment for the user 12 or to gradually administer the liquid medicine S1 over time. The liquid medicine S1 administered by the liquid medicine administration device 10 is not particularly limited. Examples of the liquid medicine S1 include liquid medicines such as antibody drugs, anticancer agents, chemotherapeutic agents, anesthetics, antibiotics, insulin, blood preparations, and nutrients.

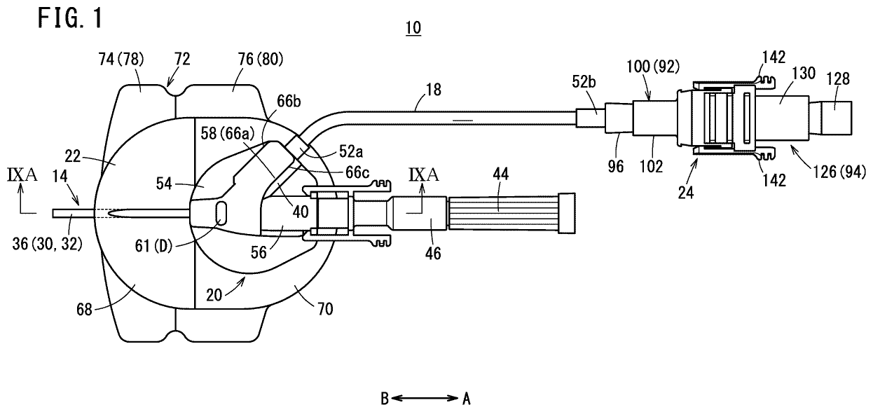

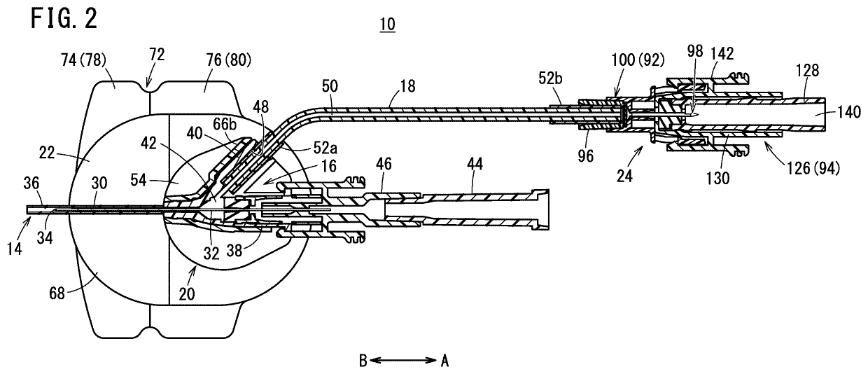

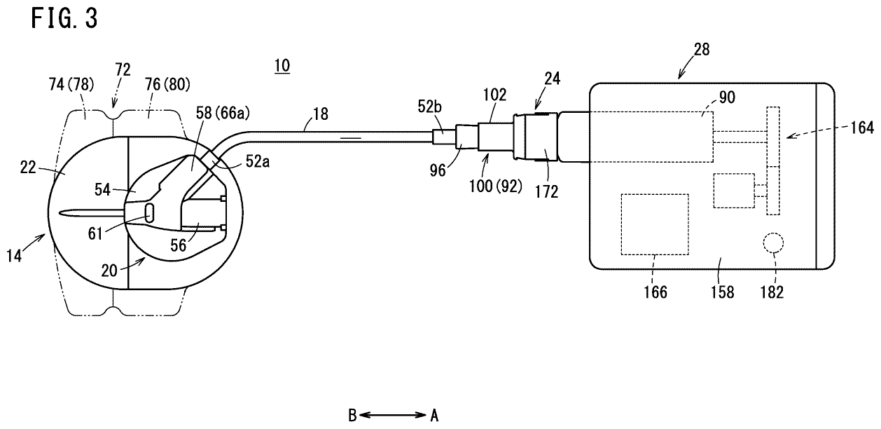

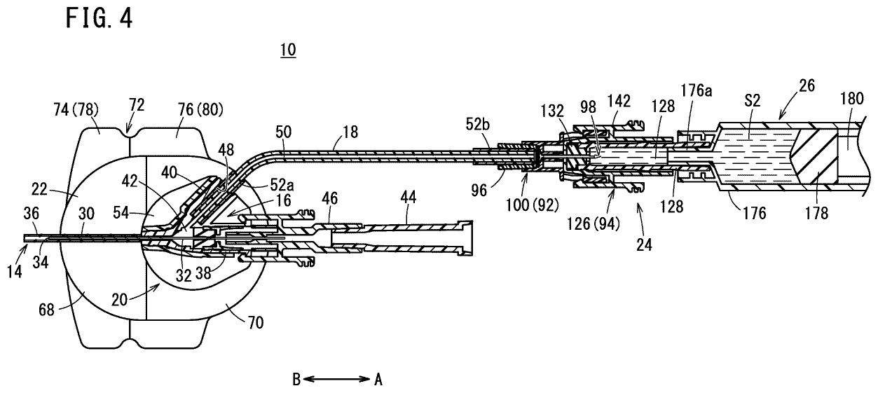

As illustrated in FIGS. 1 and 2, the liquid medicine administration device 10 includes a needle portion 14, a catheter hub 16 that holds the needle portion 14, a tube 18 through which the liquid medicine S1 can flow, the tube 18 having a distal end held by the catheter hub 16 and being connected to a catheter 30, a holder 20 that holds the catheter hub 16, an adhesive member 22 that is fixed to a back surface of the holder 20 and is to be affixed along the body surface 12a of the user 12, and a connector 24 provided at a proximal portion of the tube 18. As illustrated in FIGS. 3 and 4, the connector 24 is selectively connectable to a priming solution delivery tool 26 capable of delivering a priming solution S2 (physiological saline) charged inside from a distal portion, and a device body 28 capable of delivering the liquid medicine S1 charged inside.

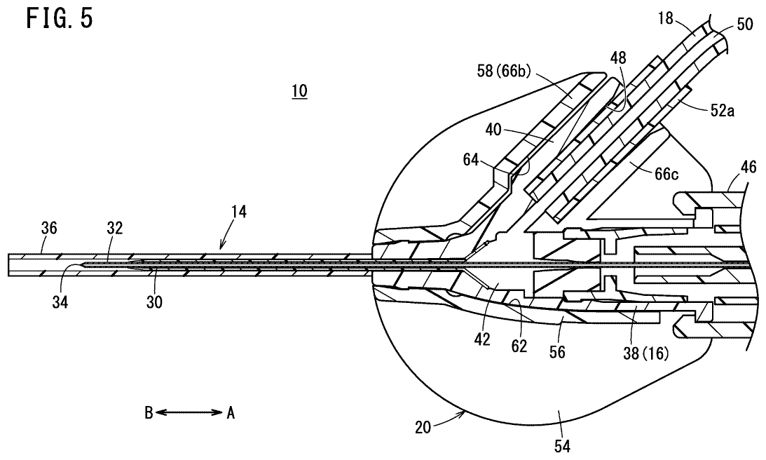

As illustrated in FIG. 5, the needle portion 14 includes the catheter 30 to be left in the body (under the skin) of the user 12 and an inner needle 32 passing through the inside of the catheter 30. The catheter 30 of the needle portion 14 is inserted into the body from the body surface 12a of the user 12 and left in place in the body. Thus, the needle portion 14 constitutes an introduction portion for the liquid medicine S1.

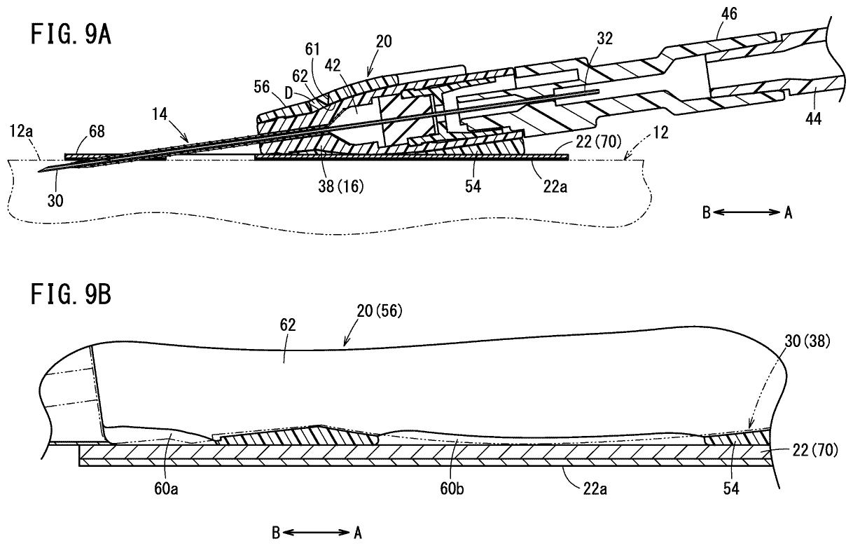

The catheter 30 is a tubular body having flexibility and including a lumen (not illustrated) through which the liquid medicine S1 can flow. The lumen of the catheter 30 communicates with a distal end opening of the catheter 30 and communicates with a space 42 in the catheter hub 16 on the proximal side (direction of arrow A) of the catheter 30. A distal portion of the catheter 30 protrudes obliquely downward from the lower surface of the catheter hub 16 (the surface facing the body surface 12a) in a distal direction (direction of arrow B) (see FIG. 9A). A proximal portion of the catheter 30 is crimped and fixed to the catheter hub 16 by, for example, a crimping pin (not illustrated).

A part of the inner needle 32 passes through the inside of the catheter 30. The distal side of the inner needle 32 is inserted into the lumen of the catheter 30. A proximal end of the inner needle 32 is inserted into the catheter hub 16 and held by a distal end of a needle hub 46 described later. The inner needle 32 is inserted into the catheter 30, and thus, a multi-needle 34 having the catheter 30 and the inner needle 32 that overlap in the radial direction is obtained. A protector 36 is attached to the multi-needle 34. The protector 36 has a tubular shape and is attached so as to cover the outer periphery of the catheter 30.

The user 12 punctures the body with the multi-needle 34 including the catheter 30 and the inner needle 32, and pulls out the inner needle 32 from the catheter 30 in a punctured state (disengages the needle hub 46 from the needle portion 14 to the proximal side (direction of arrow A)), thereby placing and leaving the catheter 30 into the body of the user 12.

As illustrated in FIG. 2, the catheter hub 16 includes a hub body 38 that supports a proximal end of the catheter 30, and a side port 40 that protrudes from a side part of the hub body 38 and to which a distal end of the tube 18 is connected. The hub body 38 extends linearly from the distal end toward the proximal end, and has a space 42 inside. The space 42 communicates with the lumen of the catheter 30 and a flow path 50 of the tube 18 connected to the side port 40. The needle hub 46 to which a grip member 44 is joined is detachably attached to the proximal end of the hub body 38.

The side port 40 protrudes and branches obliquely in the proximal direction from the vicinity of a distal end of the hub body 38. A proximal end of the side port 40 has a connection port 48 into which the distal end of the tube 18 is inserted.

The tube 18 is formed of a flexible tube having the flow path 50 through which the liquid medicine S1 can flow. Tubular joint tubes 52a and 52b are attached to the outer periphery of the tube 18 at a distal portion and a proximal portion, respectively. The distal portion of the tube 18 is fixed to the connection port 48 of the catheter hub 16 together with the joint tube 52a by an appropriate fixing method such as adhesion, welding, or crimping. Thus, the flow path 50 of the tube 18 communicates with the space 42 of the catheter hub 16 through the connection port 48.

The proximal portion of the tube 18 is inserted into a tube holder 96 of the connector 24 to be described later together with the joint tube 52b, and is fixed with an appropriate fixing method such as adhesion, welding, or crimping (see FIG. 4).

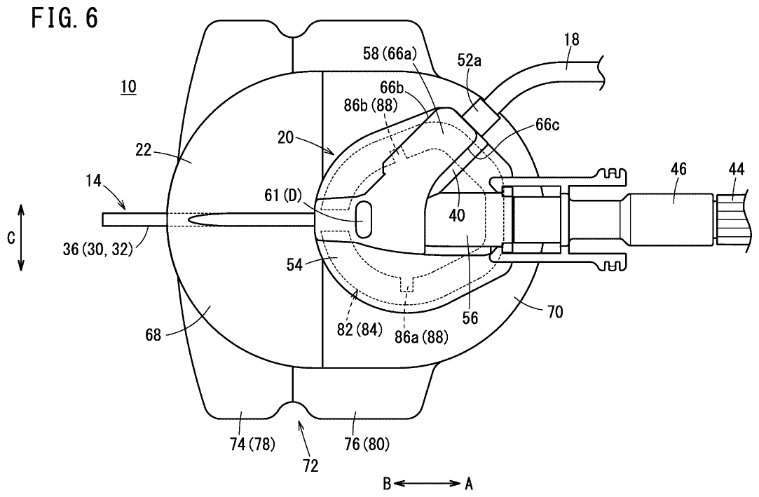

As illustrated in FIG. 6, the holder 20 includes a flat base portion 54 to which the adhesive member 22 is fixed, and a catheter holding portion 56 and a port holding portion 58 which are provided on the base portion 54 and hold the catheter hub 16.

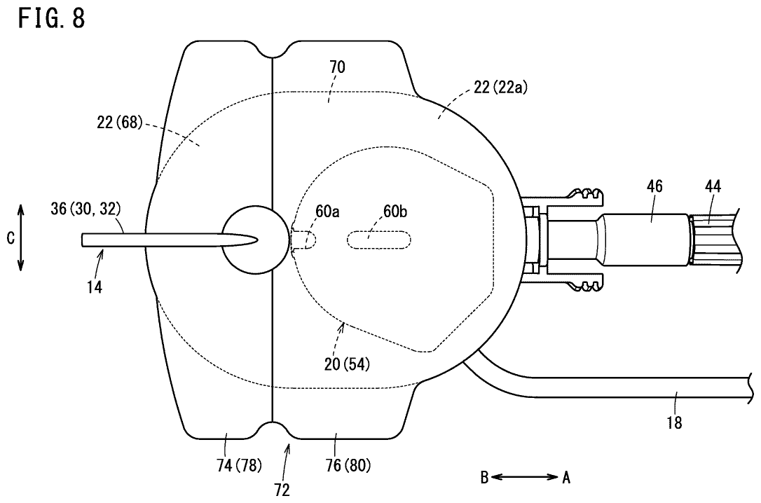

The base portion 54 is formed in a substantially circular shape in top view. The catheter holding portion 56 and the port holding portion 58 are provided on the surface of the base portion 54. The adhesive member 22 is fixed to the back surface of the base portion 54. The base portion 54 includes a notch 60a provided at its distal end and an opening 60b provided substantially at the center. The notch 60a and the opening 60b pass through the base portion 54 to the back surface (see FIGS. 8 to 9B).

As illustrated in FIGS. 6 and 9A, the catheter holding portion 56 holds the hub body 38 of the catheter hub 16. The catheter holding portion 56 is formed in a cylindrical shape and is disposed at the center of the base portion 54. The catheter holding portion 56 extends along the extending direction of the catheter 30. A distal end of the catheter holding portion 56 is disposed so as to be inclined toward the base portion 54 (see FIGS. 9A and 9B). In other words, the catheter holding portion 56 is inclined upward in the proximal direction (direction of arrow B) from the distal end. An introduction hole 61 is opened in an upper part of the catheter holding portion 56. The introduction hole 61 communicates with a first accommodation hole 62 described later.

The first accommodation hole 62 is formed inside the catheter holding portion 56 illustrated in FIGS. 5 and 9A. The first accommodation hole 62 opens to the distal end and the proximal end of the catheter holding portion 56. The first accommodation hole 62 communicates with the notch 60a and the opening 60b of the base portion 54 (see FIG. 9B). The hub body 38 of the catheter hub 16 is inserted into and held in the first accommodation hole 62 from the proximal end of the catheter holding portion 56. In this state, the lower part of the distal portion of the hub body 38 is placed in the notch 60a. That is, when the holder 20 is fixed to the body surface 12a together with the adhesive member 22, the distal portion of the hub body 38 is placed at a position close to the body surface 12a through the notch 60a (see FIG. 9A).

A part of the lower part of the hub body 38 is inserted and held in the opening 60b. When the hub body 38 of the catheter hub 16 is inserted into the first accommodation hole 62 of the catheter holding portion 56, an adhesive D is injected into the first accommodation hole 62 through the introduction hole 61. The hub body 38 is fixed to the catheter holding portion 56 by the adhesive D. The method for fixing the hub body 38 is not particularly limited, and for example, the catheter holding portion 56 and the hub body 38 may be fitted to each other.

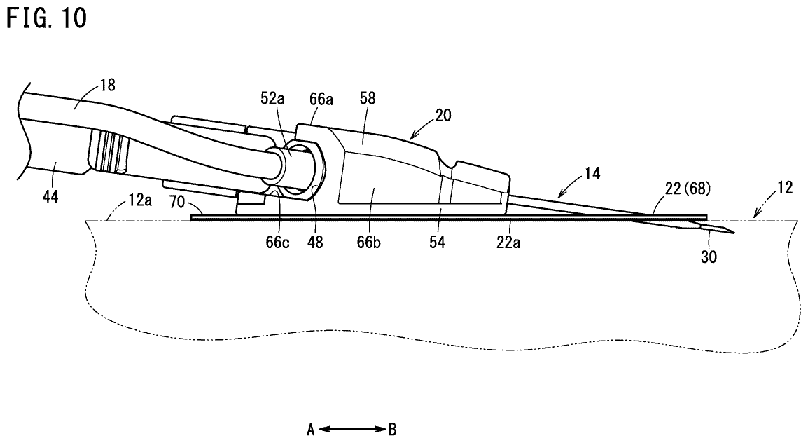

As illustrated in FIGS. 5 and 10, the port holding portion 58 holds the side port 40 of the catheter hub 16. The port holding portion 58 obliquely protrudes from the side part of the catheter holding portion 56 in the proximal direction. The port holding portion 58 is formed in a cylindrical shape and is disposed on the surface of the base portion 54. A second accommodation hole 64 is formed inside the port holding portion 58. The side port 40 of the catheter hub 16 is inserted and held in the second accommodation hole 64.

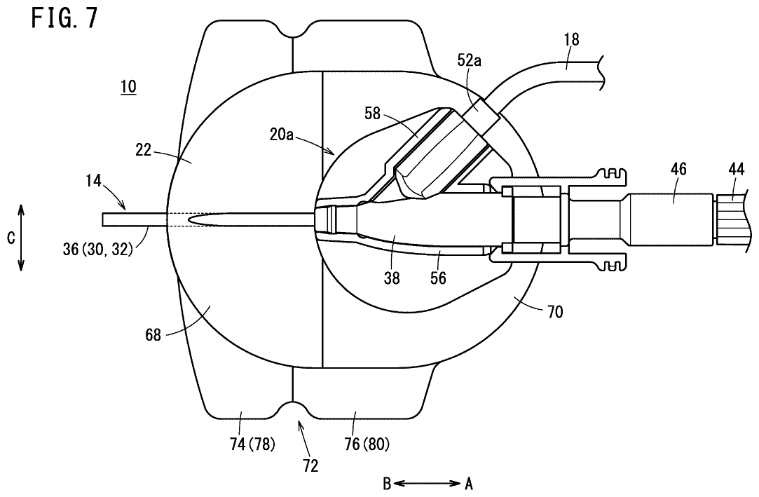

The port holding portion 58 is open on the catheter holding portion 56 side. The port holding portion 58 has a U-shaped cross section when viewed in the extending direction of the port holding portion 58. The port holding portion 58 has an upper wall 66a that covers the side port 40 from above. The upper wall 66a is in contact with the upper surface of the side port 40 and holds the side port 40. Note that, as illustrated in FIG. 7, a holder 20a not having the upper wall 66a on the port holding portion 58 may be used. In this case, the hub body 38 is fixed to the holder 20a with an adhesive or the like.

The port holding portion 58 has a side wall 66b that is orthogonal to the upper wall 66a and constitutes a side part of the port holding portion 58. The side wall 66b covers a side part of the port holding portion 58 opposite to the catheter holding portion 56. The port holding portion 58 has an opening 66c opened to the catheter holding portion 56 side. The port holding portion 58 is opened through the opening 66c on the catheter holding portion 56 side. When the side port 40 is mounted on the port holding portion 58, the side port 40 is accommodated in the second accommodation hole 64 of the port holding portion 58 through the opening 66c.

As illustrated in FIGS. 6 and 8, the adhesive member 22 has flexibility and is formed in a sheet shape wider than the base portion 54 of the holder 20 in the surface direction. The adhesive member 22 has an elliptical shape extending farther from the distal end of the base portion 54 in the distal direction (direction of arrow B). The adhesive member 22 includes a first adhesive portion 68 through which the catheter 30 is inserted, and a second adhesive portion 70 disposed at the proximal end of the first adhesive portion 68 and to which the holder 20 is fixed. The first adhesive portion 68 is disposed in the distal direction (direction of the arrow B) from the center of the adhesive member 22. The second adhesive portion 70 is disposed in the proximal direction (direction of the arrow B) from the center of the adhesive member 22. The first adhesive portion 68 and the second adhesive portion 70 are separated at substantially the center of the adhesive member 22. When viewed from above, each of the first and second adhesive portions 68 and 70 has a substantially semicircular shape.

The back surface of the adhesive member 22 is an adhesive surface 22a that is to be affixed to the body surface 12a of the user 12, and the adhesive surface 22a has adhesiveness. A release sheet 72 is affixed to the adhesive surface 22a so as to cover the adhesive surface 22a.

The release sheet 72 is formed in a thin sheet shape, and is fused by heat welding or the like so as to be overlaid on the adhesive surface 22a. The release sheet 72 includes a first sheet 74 fused to the first adhesive portion 68 and a second sheet 76 fused to the second adhesive portion 70.

The first and second sheets 74 and 76 respectively have first and second grip pieces 78 and 80 that protrude outward from the outer edge of the adhesive member 22 and can be gripped by the user 12. The first and second grip pieces 78 and 80 protrude in the width direction (direction of arrow C in FIGS. 6 and 8) orthogonal to the longitudinal direction along the distal end and the proximal end of the adhesive member 22. When the holder 20 and the adhesive member 22 are fixed to the body surface 12a of the user 12, the release sheet 72 is peeled off from the adhesive member 22 in the order of the first sheet 74 and the second sheet 76. Thus, the release sheet 72 is peeled off from the adhesive surface 22a of the adhesive member 22, so that the adhesive surface 22a is exposed.

At this time, the first and second grip pieces 78 and 80 are gripped by the user 12, and the first and second sheets 74 and 76 are peeled off from the adhesive member 22 in the width direction (direction of arrow C). The user peels off the second sheet 76 from the second adhesive portion 70 by gripping the second grip piece 80. The peeling direction of the release sheet 72 is a width direction (direction of arrow C) orthogonal to the longitudinal direction of the adhesive member 22, and the peeling direction may be either one side or the other side in the width direction.

The surface of the release sheet 72 affixed to the adhesive surface 22a of the adhesive member 22 is coated to facilitate peeling from the adhesive surface 22a.

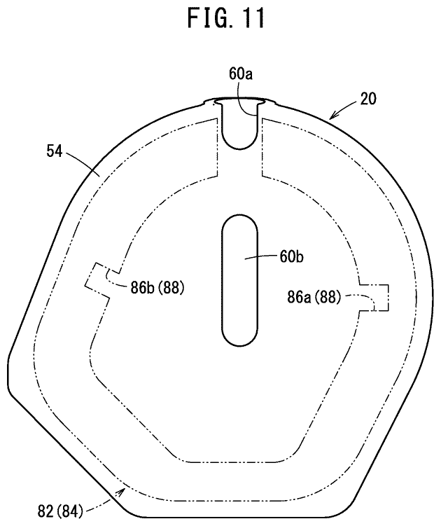

The adhesive member 22 is fixed by being fused to the back surface of the base portion 54 of the holder 20. A fusion portion 82 that bonds the holder 20 and the adhesive member 22 is provided between the back surface of the base portion 54 and the second adhesive portion 70 of the adhesive member 22 (see FIGS. 6 and 11).

As illustrated in FIG. 11, the fusion portion 82 is disposed inside from the outer edge of the holder 20 and extends along the outer periphery of the holder 20 in a substantially annular shape. The fusion portion 82 includes a general fusion portion 84 and peeling assisting portions 86a and 86b having a smaller bonding force than the general fusion portion 84. The general fusion portion 84 is formed by thermally welding the back surface of the holder 20 and the adhesive member 22. The base portion 54 of the holder 20 and the adhesive member 22 are firmly fixed with the general fusion portion 84.

When the base portion 54 of the holder 20 and the adhesive member 22 are fused, the release sheet 72 is fused to the adhesive surface 22a of the adhesive member 22 in advance. Therefore, in plan view, the release sheet 72 and the adhesive member 22 are fused at a position corresponding to the fusion portion 82. A bonding force between the release sheet 72 and the adhesive member 22 at a position corresponding to the fusion portion 82 is larger than a bonding force between the release sheet 72 and the adhesive member 22 at a position other than the fusion portion 82.

The peeling assisting portions 86a and 86b are a set of non-fusion portions 88 formed by cutting a part of the general fusion portion 84, and are disposed on the inner peripheral side of the general fusion portion 84. The peeling assisting portions 86a and 86b are recessed in a substantially rectangular shape radially outward from the inner peripheral portion of the general fusion portion 84. The shape of the peeling assisting portions 86a and 86b is not limited to the rectangular shape. Note that the peeling assisting portions 86a and 86b may not be a set of non-fusion portions, but may be a set of low fusion portions or a combination of a non-fusion portion and a low fusion portion.

The peeling assisting portions 86a and 86b are disposed at positions where a maximum load is applied when the second sheet 76 is peeled off from the second adhesive portion 70 of the adhesive member 22 along the width direction (peeling direction). Because the second sheet 76 is peeled off from the second adhesive portion 70 in the width direction, a position on the outer end in the width direction of the fusion portion 82 (the general fusion portion 84) is the position where the second sheet 76 starts to be peeled off first in the fusion portion 82. Therefore, the load (peel resistance) at the time of peeling off the release sheet 72 is the maximum.

Because the peeling direction of the second sheet 76 may be either one side or the other side in the width direction, the peeling assisting portions 86a and 86b are disposed at two positions corresponding to the two peeling directions on the outer end of the fusion portion 82 (general fusion portion 84) in the width direction. In the peeling direction of the second sheet 76, the fusion range (fusion area) of the fusion portion 82 is smaller than the fusion range of the general fusion portion 84 due to the peeling assisting portions 86a and 86b.

The peeling assisting portions 86a and 86b are disposed at positions away from the port holding portion 58 of the holder 20 in the circumferential direction of the fusion portion 82 (general fusion portion 84). The peeling assisting portion 86b is apart from the port holding portion 58 in the distal direction.

Note that the peeling assisting portions 86a and 86b are not limited to the non-fusion portions 88 that are not fused to the base portion 54 of the holder 20 and the adhesive member 22 by cutting out a part of the general fusion portion 84. For example, a fusion portion (second fusion portion) fused with a bonding force smaller than the bonding force of the general fusion portion 84 may be provided in a part of the fusion portion 84. When the holder 20 and the adhesive member 22 are fused by thermal welding, it is possible to provide the fusion portion 82 having a smaller bonding force than that of the general fusion portion 84 in a part of the fusion portion 82 by lowering a welding temperature of a part of the portion to be fused.

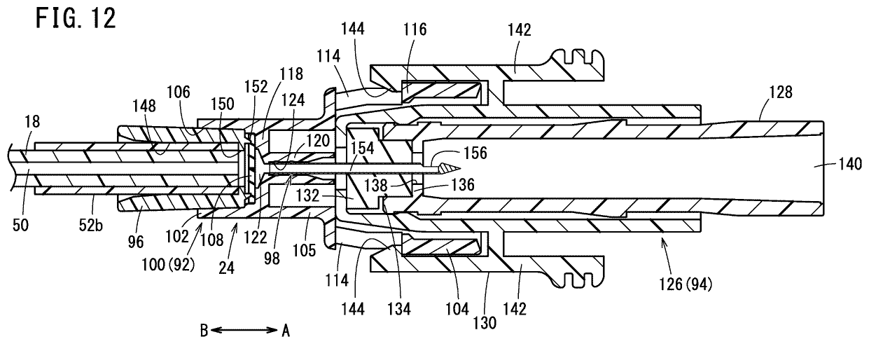

As illustrated in FIGS. 1 and 12, the connector 24 includes a first connection portion 92 connectable to a prefilled syringe 90 of the device body 28 described later, a second connection portion 94 connectable to the priming solution delivery tool 26 capable of delivering the priming solution S2, the tube holder 96 holding a proximal end of the tube 18 and attached to the first connection portion 92, and a hollow needle 98 held by the first connection portion 92 and protruding in the proximal direction.

The first connection portion 92 includes a first connection member 100 connected to the proximal portion of the tube 18 via the tube holder 96.

The first connection member 100 includes a distal connection portion 102 formed at the distal end and holding the proximal end of the tube 18 together with the tube holder 96, a proximal connection portion 104 formed at the proximal end and selectively connected to the device body 28 or the second connection portion 94, and an intermediate portion 105 located between the distal connection portion 102 and the proximal connection portion 104.

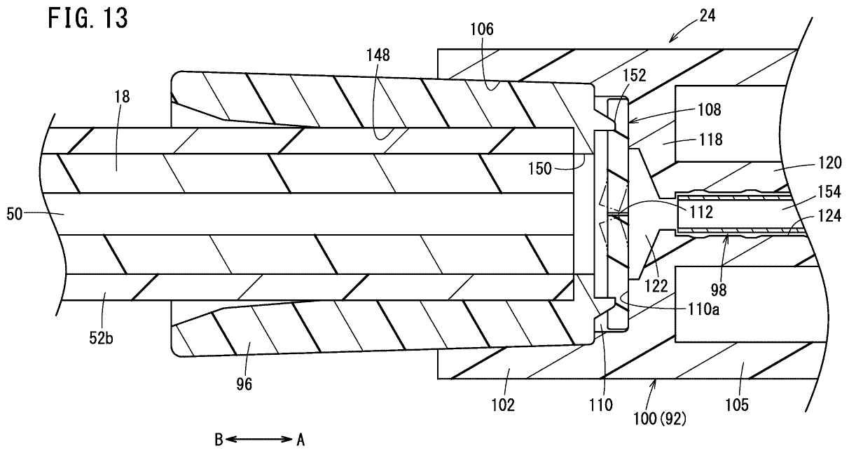

As illustrated in FIG. 13, the distal connection portion 102 is formed in a cylindrical shape opened toward the distal end. The distal connection portion 102 includes a holder hole 106 into which the tube holder 96 is inserted and held and a valve hole 110 which is formed at a proximal end of the holder hole 106 and in which a valve body 108 is accommodated. The holder hole 106 gradually decreases in diameter in the proximal direction (direction of arrow A) from the distal end, and the tube holder 96 is held inside.

The valve hole 110 has a circular cross section with a diameter smaller than that of the holder hole 106. A proximal end face 110a of the valve hole 110 illustrated in FIG. 13 is a flat surface orthogonal to the axial direction of the first connection member 100. The valve body 108 is in contact with the proximal end face 110a, and a communication path 122 to be described later opens at the center of the proximal end face 110a. As a result, the dead volume in the first connection member 100 can be reduced.

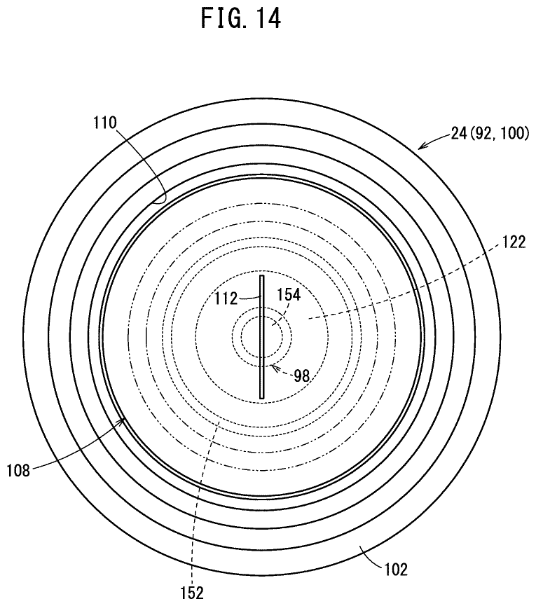

As illustrated in FIGS. 13 and 14, the valve body 108 is formed in a disk shape from an elastic material such as silicone rubber. The valve body 108 is formed to have a constant thickness with an axial dimension (thickness dimension) smaller than a radial dimension.

A slit-shaped opening and closing hole 112 is formed at the center of the valve body 108. The shape of the opening and closing hole 112 is straight as viewed in the axial direction of the valve body 108 illustrated in FIG. 14. The opening and closing hole 112 linearly extends radially outward from the central part of the valve body 108 and passes through the valve body 108 in the thickness direction. Note that the opening and closing hole 112 is not limited to have a straight line shape (slit shape), and may be formed to have a cross shape, for example.

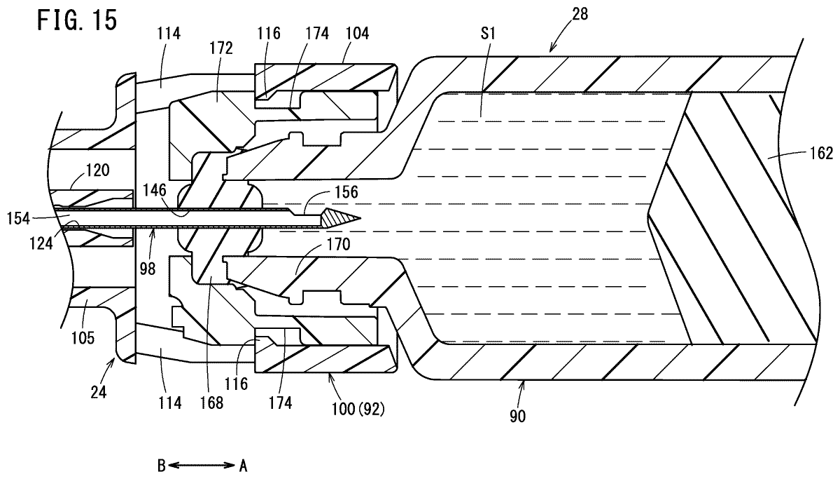

A pressure of the priming solution S2 or the liquid medicine S1 supplied from the hollow needle 98 to be described later to the communication path 122 is applied to the proximal end face of the valve body 108. When pressure is applied, the peripheral edge of the opening and closing hole 112 is pushed in the distal direction, so that the opening and closing hole 112 is elastically deformed in the distal direction (see the shape indicated in two-dot chain line in FIG. 13). As a result, the opening and closing hole 112 is opened, and in the connector 24, the communication path 122 of the first connection member 100 and the tube 18 held by the tube holder 96 communicate with each other through the opening and closing hole 112. The opening pressure applied to the valve body 108 by the liquid medicine S1 for opening the opening and closing hole 112 is lower than the pressure applied to the valve body 108 by the driving force when the device body 28 is driven. Thus, the opening and closing hole 112 of the valve body 108 is opened with a low pressure, whereby it is possible to prevent an increase in the dead volume due to deformation of a first gasket 162 (see FIG. 15) by the back pressure from the valve body 108 when the administration of the liquid medicine S1 is completed.

When no pressure is applied to the proximal end face of the valve body 108, the opening and closing hole 112 is closed, and communication between the communication path 122 of the first connection member 100 and the tube 18 is blocked by the valve body 108.

As illustrated in FIG. 12, the proximal connection portion 104 is provided at the proximal end of the intermediate portion 105 and has a cylindrical shape opened in the proximal direction (direction of arrow A). The proximal connection portion 104 has a larger diameter than the distal connection portion 102 and the intermediate portion 105. A pair of engagement holes 114 opened in a rectangular shape is provided in an outer peripheral wall of the proximal connection portion 104. The pair of engagement holes 114 passes through the outer peripheral wall in the radial direction. The pair of engagement holes 114 faces each other about the axis of the proximal connection portion 104. When the second connection portion 94 is connected to the proximal connection portion 104, arms 142 of the second connection portion 94 described later are engaged with the engagement holes 114.

The outer peripheral wall of the proximal connection portion 104 includes a pair of connector tabs 116 protruding radially inward from the inner peripheral surface. As illustrated in FIG. 15, when the distal portion of the prefilled syringe 90 of the device body 28 is connected to the first connection member 100, the connector tabs 116 are engaged with engagement recesses 174 formed in the distal portion. Thus, the device body 28 is connected to the proximal end of the first connection member 100 via the proximal connection portion 104.

The intermediate portion 105 is formed in a cylindrical shape and includes a separation wall 118 formed at a distal end, a support portion 120 protruding in the proximal direction (direction of arrow A) from the center of the separation wall 118, and the communication path 122 formed inside the separation wall 118. The communication path 122 passes through the central part of the separation wall 118 and communicates with the valve hole 110.

The support portion 120 extends from the separation wall 118 to the distal end of the proximal connection portion 104 along the axial direction, and a needle fitting hole 124 is formed therein. A distal end of the needle fitting hole 124 communicates with the communication path 122. The hollow needle 98 is fitted and held in the needle fitting hole 124. Note that the hollow needle 98 may be held by fixing with an adhesive or insert molding.

The second connection portion 94 includes a second connection member 126 detachably attached to the proximal portion of the first connection member 100. The second connection member 126 includes a first cylindrical body 128 to which the distal portion of the priming solution delivery tool 26 is connected, a second cylindrical body 130 that covers the outside of the first cylindrical body 128 and is engaged with the first connection member 100, and a cap 132 held at the distal ends of the first and second cylindrical bodies 128 and 130.

The first cylindrical body 128 is formed in a cylindrical shape, and is integrally held into the second cylindrical body 130 by press fitting. The first cylindrical body 128 includes a holding end 134 provided at the distal end and capable of holding the cap 132, and an intermediate wall 136 located at the proximal end of the holding end 134.

The holding end 134 is open toward the distal end, and the proximal portion of the cap 132 is inserted into the holding end 134, by which the cap 132 is held by the holding end 134. The outer peripheral surface of the holding end 134 is integrally held by being engaged with the inner peripheral surface of the second cylindrical body 130. The intermediate wall 136 is orthogonal to the axial direction of the first cylindrical body 128, and an insertion hole 138 is formed at the center of the intermediate wall 136. The insertion hole 138 passes through the intermediate wall 136 in the axial direction.

The proximal end of the first cylindrical body 128 has a fitting hole 140 opened in the proximal direction (direction of arrow A), and the fitting hole 140 is formed in a tapered shape from the proximal end toward the distal end. A distal nozzle portion 176a of a barrel 176 of the priming solution delivery tool 26 is inserted into the fitting hole 140, and a distal portion of the barrel 176 is connected to the proximal end of the first cylindrical body 128. The distal nozzle portion 176a of the barrel 176 is tapered in the distal direction, and thus, is fitted to the fitting hole 140 in a tapered manner.

The second cylindrical body 130 is formed in a cylindrical shape having a larger diameter than the first cylindrical body 128. The distal end of the second cylindrical body 130 is formed in a tapered shape in the distal direction. The distal end of the second cylindrical body 130 can be inserted into the proximal connection portion 104 of the first connection member 100. A pair of arms 142 is provided on the outer periphery of the second cylindrical body 130.

The arms 142 are elastically deformable. The arms 142 are disposed at symmetrical positions with respect to the axial center of the second cylindrical body 130. The arms 142 are disposed on the radially outer side of and parallel to the outer peripheral surface of the second cylindrical body 130, and are supported by the outer peripheral surface at substantially central parts along the extending direction. The arms 142 can tilt about the substantially central parts. Each of the arms 142 has, on the distal end thereof, an engagement portion 144 protruding radially inward. The engagement portion 144 has a claw shape tapered radially inward.

The engagement portion 144 can approach or move away from the first connection member 100 by the tilting movement of the arm 142.

When the second connection member 126 is connected to the proximal connection portion 104 of the first connection member 100, the engagement portions 144 of the arms 142 are engaged with the engagement holes 114 of the proximal connection portion 104 by elasticity. Thus, the second connection member 126 is connected to the proximal end of the first connection member 100. The first connection member 100 and the second connection member 126 are connected with the relative movement in the axial direction (direction of arrow A, B) and the rotational direction being restricted.

The cap 132 is formed of, for example, an elastic material such as rubber and has a circular cross section as viewed in the axial direction. A needle insertion hole 146 into which the hollow needle 98 is inserted is opened in the axial center of the cap 132 so as to penetrate the cap 132. The cap 132 is held between the distal end of the first cylindrical body 128 and the distal end of the second cylindrical body 130.

As illustrated in FIGS. 12 and 13, the tube holder 96 has a bottomed cylindrical shape with its distal end opened. The outer peripheral surface of the tube holder 96 is formed in a tapered shape in the proximal direction from the distal end, and the tube holder 96 is fitted and fixed by being inserted into the holder hole 106 of the first connection member 100.

A tube hole 148 opened in the distal direction is formed inside the tube holder 96 (see FIG. 12). The proximal portion of the tube 18 to which the joint tube 52b is attached is inserted into the tube hole 148, and is fixed with an appropriate fixing method such as adhesion, welding, or crimping.

The tube holder 96 has, at the proximal end thereof, a through hole 150 formed at the center, penetrating in the axial direction, and communicating with the flow path 50 of the tube 18, and an annular valve seat 152 disposed radially outside the through hole 150.

As illustrated in FIG. 13, the valve seat 152 protrudes from the proximal end of the tube holder 96 in the proximal direction (direction of arrow A). The distal end face of the valve body 108 is in contact with the proximal end of the valve seat 152. The valve body 108 is held between the valve seat 152 of the tube holder 96 and the distal end face of the separation wall 118 of the first connection member 100. The valve seat 152 is disposed radially outside the opening and closing hole 112 of the valve body 108 and is in contact with the valve body 108 (see FIGS. 13 and 14). In this state, as illustrated in FIG. 13, the proximal portion of the valve seat 152 comes in contact with and slightly bites into the distal end face of the valve body 108 in the proximal direction (direction of arrow A), so that the valve body 108 is held in a state of being tightened between the proximal end face 110a of the valve hole 110 and the valve seat 152 (see FIG. 13).

As illustrated in FIGS. 12 and 15, the hollow needle 98 is a needle body formed of metal and having a needle hole 154 therein, and connects the device body 28 or the second connection member 126 and the communication path 122 of the first connection member 100. When the hollow needle 98 is fitted into the needle fitting hole 124 of the support portion 120 of the first connection member 100, the hollow needle 98 is held at the axial center of the first connection member 100. Note that the hollow needle 98 may be held by fixing with an adhesive or insert molding, instead of fitting. Further, the hollow needle 98 may be made of plastic and integrally molded. The distal end of the hollow needle 98 communicates with the communication path 122 via the needle hole 154.

A proximal end of the hollow needle 98 is formed in a sharp shape, and has a proximal end opening 156 communicating with the needle hole 154. The proximal end of the hollow needle 98 is exposed from the proximal end of the support portion 120 by a predetermined length in the proximal direction (direction of arrow A). The proximal end of the hollow needle 98 is accommodated in the proximal connection portion 104 and does not protrude from the proximal end of the proximal connection portion 104 in the proximal direction (direction of arrow A). The outer periphery of the hollow needle 98 is enclosed by the proximal connection portion 104 (see FIG. 12).

As illustrated in FIG. 12, when the second connection member 126 is connected to the first connection member 100, the proximal end of the hollow needle 98 exposed inside the proximal connection portion 104 is inserted into the insertion hole 138 of the first cylindrical body 128 through the needle insertion hole 146 of the cap 132. Thus, the proximal end of the hollow needle 98 protrudes in the proximal direction of the cap 132, and the space between the hollow needle 98 and the first cylindrical body 128 is sealed by the cap 132. The proximal end opening 156 of the hollow needle 98 is located in and communicates with the first cylindrical body 128, whereby the communication path 122 of the first connection member 100 and the inside of the first cylindrical body 128 are connected and communicates with each other by the hollow needle 98.

As illustrated in FIG. 3, the device body 28 has a function of storing the liquid medicine S1 and delivering the liquid medicine S1 from the distal portion at an appropriate timing, and includes a case 158 that has an accommodation space (not illustrated) for accommodating the components.

The case 158 includes therein the prefilled syringe 90 that stores the liquid medicine S1, a movement mechanism 164 that moves the first gasket 162 in the prefilled syringe 90, a control unit 166 that controls the driving of the movement mechanism 164, and a power supply unit (not illustrated) that can supply power to each component.

As illustrated in FIG. 15, the prefilled syringe 90 is formed in a cylindrical shape along the axial direction (direction of arrow A, B), and is filled with the liquid medicine S1. The distal portion of the prefilled syringe 90 protrudes in the axial direction (direction of arrow B) by a predetermined length from the distal end of the case 158 and is exposed to the outside (see FIG. 3).

A delivery nozzle portion 170 having a reduced diameter and sealed with a packing 168 is provided at a distal portion of the prefilled syringe 90. The packing 168 is held between the distal end of a tubular cover member 172 provided to cover the outer peripheral side of the delivery nozzle portion 170 and the distal end of the delivery nozzle portion 170. Thus, the liquid medicine S1 is enclosed and retained in the prefilled syringe 90 by the packing 168. Engagement recesses 174 recessed radially inward are formed in the outer periphery of the cover member 172. The connector tabs 116 of the second connection member 126 can be engaged with the engagement recesses 174.

As illustrated in FIG. 4, the priming solution delivery tool 26 includes a cylindrical barrel 176, a second gasket 178 movably provided inside the barrel 176, and a rod 180 to which the second gasket 178 is connected. The proximal end of the rod 180 protrudes in the proximal direction from the proximal end of the barrel 176. The distal nozzle portion 176a of the barrel 176 has a smaller diameter than the barrel 176 and is formed in a tapered shape toward the distal end. In the barrel 176, the priming solution S2 is filled between the distal nozzle portion 176a and the second gasket 178. The distal nozzle portion 176a of the barrel 176 is fitted in a tapered manner to the fitting hole 140 of the first cylindrical body 128 of the connector 24. Thus, the distal nozzle portion 176a of the barrel 176 and the fitting hole 140 of the first cylindrical body 128 are liquid-tightly connected.

The second gasket 178 is connected to the distal end of the rod 180, and when the user 12 pushes the rod 180 in the distal direction, the second gasket 178 moves in the distal direction (direction of arrow B) along the barrel 176. As a result, the priming solution S2 inside the barrel 176 is pushed in the distal direction by the second gasket 178 and delivered from the opening of the distal portion (distal nozzle portion 176a) of the barrel 176.

Next, the administration of the liquid medicine S1 to the user 12 using the liquid medicine administration device 10 will be described. When the liquid medicine administration device 10 is used, the needle portion 14 is left in place on the body surface 12a of the user 12, the priming solution delivery tool 26 is connected to fill the inside of the catheter 30 with the priming solution S2, and then the device body 28 is connected to automatically administer the liquid medicine S1 to the user 12.

First, the first priming for filling the needle portion 14 and the tube 18 with the priming solution S2 is performed.

The priming solution delivery tool 26 is connected to the proximal end of the second connection member 126 of the connector 24 of the liquid medicine administration device 10 illustrated in FIGS. 1 and 2 (see FIG. 4). The distal nozzle portion 176a of the barrel 176 of the priming solution delivery tool 26 is inserted into the fitting hole 140 from the proximal end of the first cylindrical body 128 of the connector 24. The distal nozzle portion 176a of the barrel 176 is fitted into the fitting hole 140 of the first cylindrical body 128 and is held in a liquid-tight manner. As a result, the inside of the barrel 176 and the inside of the first cylindrical body 128 communicate with each other. The priming solution S2 in the barrel 176 flows from the distal nozzle portion 176a of the barrel 176 into the fitting hole 140.

When the user 12 pushes the rod 180 of the priming solution delivery tool 26 in the distal direction (direction of arrow B), the second gasket 178 moves in the distal direction along the barrel 176, and accordingly, the priming solution S2 in the barrel 176 is delivered to the fitting hole 140 of the first cylindrical body 128 from the distal nozzle portion 176a of the barrel 176.

The priming solution S2 in the first cylindrical body 128 flows in the distal direction through the proximal end opening 156 of the hollow needle 98 and the needle hole 154 and is introduced into the communication path 122.

The proximal end face of the valve body 108 is pressed in the distal direction (direction of arrow B) by the priming solution S2 supplied into the communication path 122. The central part of the valve body 108 is elastically deformed so as to be bent in the distal direction around the opening and closing hole 112, and accordingly, the opening and closing hole 112 is opened. The priming solution S2 passes through the opening and closing hole 112 from the communication path 122 and flows toward the flow path 50 of the tube 18 through the valve hole 110.

The priming solution S2 flows into the space 42 of the catheter 30 from the flow path 50 of the tube 18 through the side port 40, and then, the priming solution S2 circulates into the space 42 of the catheter 30. Thus, the tube 18, the space 42, and the inside of the catheter 30 are filled with the priming solution S2, and the first priming is completed.

When the supply of the priming solution S2 by the priming solution delivery tool 26 is stopped, the pressure is not applied to the proximal end face of the valve body 108. Therefore, the opened opening and closing hole 112 returns to the original shape by the elasticity of the valve body 108, and the opening and closing hole 112 is closed.

Next, the needle portion 14 is left in place in the body of the user 12. The user 12 grips and positions the needle portion 14 (multi-needle 34) at a desired position on the body surface 12a, and then removes the protector 36 from the multi-needle 34 in the distal direction.

After puncturing the body with the distal end of the multi-needle 34, the user 12 grips the first grip piece 78 of the first sheet 74 (release sheet 72) and peels off the first sheet 74 from the adhesive member 22 in the width direction. Because the first sheet 74 is peeled off from the back surface of the adhesive member 22, the adhesive surface 22a of the first adhesive portion 68 is exposed. The adhesive surface 22a of the first adhesive portion 68 is affixed along the body surface 12a of the user 12 by adhesive force. Thus, the distal end of the needle portion 14 is fixed to the body surface 12a in a state of puncturing the user 12. That is, only the distal side (first adhesive portion 68) of the adhesive member 22 holding the needle portion 14 is fixed to the body surface 12a.

Next, the user 12 pulls out the needle hub 46 and the grip member 44 in the proximal direction (direction of arrow A) from the needle portion 14 to release the engagement between the needle hub 46 and the catheter hub 16, and pulls out the inner needle 32 held by the needle hub 46 from the catheter hub 16. During this operation, the catheter 30 of the needle portion 14 is fixed to the body surface 12a, whereby the inner needle 32 is removed from the inside of the catheter 30, and only the catheter 30 is left in place in the body.

After the catheter 30 is left in place, the user 12 grips the second grip piece 80 of the second sheet 76 and peels off the second sheet 76 from the adhesive member 22 in the width direction. The second sheet 76 is peeled off from the adhesive surface 22a from one side to the other side in the width direction of the adhesive member 22. During this process, the bonding force between the release sheet 72 and the adhesive member 22 is larger at the position corresponding to the fusion portion 82, but due to the peeling assisting portions 86a and 86b provided at the outer end in the width direction of the fusion portion 82, the fusion area at the outer end in the width direction is smaller than that of the general fusion portion 84. Thus, the load (peel resistance) required for peeling decreases.

Therefore, even if the bonding force of the release sheet 72 at the position corresponding to the fusion portion 82 increases due to the fusion between the holder 20 and the adhesive member 22, the second sheet 76 can be easily peeled off from the adhesive member 22.

The second sheet 76 is peeled off from the back surface of the adhesive member 22, by which the adhesive surface 22a of the second adhesive portion 70 is exposed. The adhesive surface 22a of the second adhesive portion 70 is affixed along the body surface 12a of the user 12 by adhesive force. In addition to the first adhesive portion 68 of the adhesive member 22, the second adhesive portion 70 is fixed to the body surface 12a. That is, the entire adhesive surface 22a of the adhesive member 22 is affixed and fixed on the body surface 12a. As a result, the holder 20 holding the catheter hub 16 is fixed to the body surface 12a of the user 12 together with the adhesive member 22.

Next, the second priming for filling again the needle portion 14 and the tube 18 with the priming solution S2 is performed.

The second priming is performed using the priming solution S2 remaining in the barrel 176 used in the first priming. Because the second priming is the same as the first priming, the detailed description thereof will be omitted.

By performing the second priming described above, backflow of blood, cells, and the like from the body surface 12a punctured by the catheter 30 is suitably prevented by the priming solution S2 charged in the tube 18 and the catheter 30. Thus, clogging of the catheter 30 and the tube 18 due to coagulation of the blood and the like is prevented.

After the second priming is completed, the priming solution delivery tool 26 is removed together with the second connection member 126.

The user 12 pushes the proximal ends of the arms 142 of the second connection member 126 radially inward. The proximal ends of the arms 142 are elastically deformed radially inward, and accordingly, the distal ends of the arms 142 move radially outward, so that the engagement portions 144 are disengaged from the engagement holes 114. Thus, the connection of the second connection member 126 to the first connection member 100 is released.

The priming solution delivery tool 26 is pulled out together with the second connection member 126 in the proximal direction (direction of arrow A), whereby the second connection member 126 and the priming solution delivery tool 26 are removed from the proximal connection portion 104 of the first connection member 100.

Next, as illustrated in FIG. 3, the device body 28 is attached to the connector 24 to administer the liquid medicine S1.

As illustrated in FIG. 15, the distal portion of the prefilled syringe 90 in the device body 28 is inserted into the proximal connection portion 104 of the first connection member 100 of the connector 24. Thus, the axial center of the packing 168 is punctured with the proximal end of the hollow needle 98. The proximal end of the hollow needle 98 is inserted into the delivery nozzle portion 170, and the connector tabs 116 are engaged with the engagement recesses 174 of the cover member 172. As a result, the distal portion of the device body 28 is connected to the proximal connection portion 104 of the connector 24.

After the user 12 turns on a power switch 182 provided in the case 158 to activate the device body 28, the control unit 166 measures the timing of starting the administration of the liquid medicine by the liquid medicine administration device 10. At the administration start timing, the movement mechanism 164 is driven by a control signal from the control unit 166 to move the first gasket 162 in the distal direction. With the movement of the first gasket 162, the liquid medicine S1 in the prefilled syringe 90 is expelled in the distal direction, and the liquid medicine S1 flows to the communication path 122 of the first connection member 100 through the proximal end opening 156 and the needle hole 154 of the hollow needle 98 located in the delivery nozzle portion 170.

The liquid medicine S1 supplied into the communication path 122 pushes the proximal end face of the valve body 108 in the distal direction, so that the central part of the valve body 108 elastically deforms in the distal direction about the opening and closing hole 112. Thus, the opening and closing hole 112 of the valve body 108 is opened. At this time, the opening pressure for opening the opening and closing hole 112 is lower than the pressure applied from the liquid medicine S1 to the proximal end face of the valve body 108 by the driving force of the device body 28.

The liquid medicine S1 passes through the opening and closing hole 112 and the valve hole 110 and flows into the space 42 of the catheter hub 16 through the flow path 50 of the tube 18. The liquid medicine S1 is administered from the space 42 into the body of the user 12 through the lumen of the catheter 30.

When the administration of the liquid medicine S1 from the device body 28 into the body is completed and the flow of the liquid medicine S1 is stopped, the valve body 108 returns to the original shape by elasticity, and the opening and closing hole 112 is closed. As a result, the administration of the liquid medicine S1 into the body of the user 12 is completed.

As described above, the first embodiment includes: the catheter hub 16 that holds the proximal end of the catheter 30; and the connector 24 to which the device body 28 is attached, the connector 24 being provided at the proximal end of the tube 18. The disk-shaped valve body 108 having the slit-shaped opening and closing hole 112 that is opened when the liquid medicine S1 is delivered is provided in the flow path through which the liquid medicine S1 flows between the catheter hub 16 and the proximal end of the connector 24.

With this configuration, the valve body 108 can reliably prevent leakage of the liquid medicine S1 from the device body 28 to the catheter 30 due to pressure in the distal direction generated during connection of the device body 28 to the connector 24 when the catheter 30 is not left in place in the body of the user 12. This configuration can also reliably prevent leakage of the liquid medicine S1 by the valve body 108 before administration when the catheter 30 is left in place in the body. When the catheter 30 is left in place in the body and the liquid medicine S1 is delivered from the device body 28, it is possible to deliver the liquid medicine S1 by driving the movement mechanism 164 of the device body 28 with a small driving force and easily opening the slit-shaped opening and closing hole 112 with a small force.

Because the valve body 108 is disposed between the proximal end of the tube 18 and the connector 24, the delivery state of the liquid medicine S1 to the catheter 30 can be switched by the valve body 108 between the device body 28 connected to the connector 24 and the tube 18 connected to the catheter 30.

The priming solution delivery tool 26 capable of delivering the priming solution S2 is selectively connected to the connector 24, the annular valve seat 152 disposed in the connector 24 is in contact with the valve body 108 on the radially outer side of the opening and closing hole 112, and the valve body 108 is held between the proximal end face 110a (see FIG. 13) of the valve hole 110 of the first connection member 100 and the valve seat 152. This configuration can suppress entry of body fluid (blood) to the connector 24 side through the space between the proximal end face 110a of the valve hole 110 and both the valve seat 152 and the valve body 108 due to the back pressure applied to the distal end face of the valve body 108, when the priming solution delivery tool 26 is removed from the connector 24 in a state where the catheter 30 is left in place in the body.

When the liquid medicine S1 is administered from the device body 28, the opening pressure applied to the valve body 108 is set to be lower than the pressure applied to the valve body 108 by the driving force when the device body 28 is driven. With this configuration, the opening and closing hole 112 can be reliably opened when the catheter 30 is left in place in the body and the liquid medicine S1 is delivered. In addition, when the connector 24 and the prefilled syringe 90 are connected in a state where the catheter 30 is not left in place in the body or when the catheter 30 is left in place in the body, the opening and closing hole 112 of the valve body 108 is closed, so that leakage of the liquid medicine S1 due to a change in internal pressure of the liquid medicine S1 caused by a change in atmospheric pressure, a change in temperature, or the like can be reliably prevented.

The first embodiment includes the connector 24 disposed at the proximal portion of the tube 18, and the hollow needle 98 protruding in the proximal direction is provided inside the connector 24. The connector 24 includes the first connection portion 92 connectable to the prefilled syringe 90 and the second connection portion 94 connectable to the priming solution delivery tool 26, the second connection portion 94 being provided at a position different from the first connection portion 92.

With this configuration, the proximal end of the hollow needle 98 is accommodated inside the connector 24. Thus, it is possible to prevent the user 12 from being erroneously punctured with the hollow needle 98 when connecting the priming solution delivery tool 26 to the second connection portion 94 to perform priming, and then, removing the priming solution delivery tool 26 and the second connection portion 94. When the prefilled syringe 90 is connected to the first connection portion 92 of the connector 24, pressure in the distal direction is not applied by the prefilled syringe 90, so that leakage of the liquid medicine S1 in the distal direction is prevented.

Because the connector 24 can be connected to the priming solution delivery tool 26 via the second connection portion 94, priming can be performed by the liquid medicine administration device 10 before the prefilled syringe 90 of the device body 28 is connected.

The first connection portion 92 is provided in the first connection member 100 connected to the proximal portion of the tube 18, and the second connection portion 94 is provided in the second connection member 126 detachably attached to the proximal portion of the first connection member 100. With this configuration, after the delivery of the priming solution S2 by the priming solution delivery tool 26 is completed, the priming solution delivery tool 26 can be removed from the first connection member 100 together with the second connection member 126, and the prefilled syringe 90 can be connected to the first connection portion 92 of the first connection member 100 to administer the liquid medicine S1.

The second connection member 126 includes the first cylindrical body 128 having a cylindrical shape and connected to the distal portion of the priming solution delivery tool 26 and the second cylindrical body 130 that covers the outside of the first cylindrical body 128 and is engaged with the first connection member 100. With this configuration, the distal nozzle portion 176a of the barrel 176 can be liquid-tightly connected to the fitting hole 140 of the first cylindrical body 128, and can be detachably connected to the first connection member 100 by the second cylindrical body 130.

The second cylindrical body 130 of the second connection member 126 has the arms 142 that can tilt in a direction approaching or away from the first connection member 100, and the engagement portions 144 provided at the distal ends of the arms 142 are engaged with the engagement holes 114 formed in the outer peripheral surface of the first connection member 100. With this configuration, the priming solution delivery tool 26 can be connected to the first connection portion 92 of the first connection member 100 via the second connection member 126, and the connection of the second connection member 126 to the connector 24 (first connection member 100) can be easily released by tilting the arms 142 to disengage the engagement portions 144 from the engagement holes 114.

The first connection portion 92 has the tapered fitting hole 140 into which the distal nozzle portion 176a of the priming solution delivery tool 26 can be inserted and fitted, the fitting hole 140 facing the distal end of the hollow needle 98. With this configuration, the distal nozzle portion 176a of the barrel 176 is fitted into the fitting hole 140, by which the distal nozzle portion 176a of the barrel 176 and the fitting hole 140 are fitted to each other in a liquid-tight manner. Thus, the priming solution delivery tool 26 and the connector 24 can be satisfactorily connected.

The cap 132 that is held at the distal ends of the first cylindrical body 128 and the second cylindrical body 130 is provided, the cap 132 being punctured by the hollow needle 98. With this configuration, the distal end of the first cylindrical body 128 and the distal end of the second cylindrical body 130 can be connected in a liquid-tight manner, so that the leakage of the priming solution S2 can be prevented by the cap 132.

The first embodiment includes the catheter hub 16 for holding the proximal end of the catheter 30, the holder 20 for holding the catheter hub 16, and the sheet-shaped adhesive member 22 fixed to the back surface of the holder 20 and to be affixed along the body surface 12a of the user 12. With this configuration, when the catheter 30 is left in place in the body of the user 12, the catheter hub 16 is reliably fixed to the adhesive member 22 by the holder 20, and thus, it is possible to prevent the catheter hub 16 from relatively moving with respect to the catheter 30 due to, for example, the body movement of the user 12. Therefore, it is possible to prevent deformation of the catheter 30 due to, for example, the body movement of the user 12 and to prevent the dislodgement of the catheter 30 from the body.

The holder 20 includes the flat base portion 54 to which the adhesive member 22 is fixed, and the cylindrical catheter holding portion 56 into which the distal portion of the catheter hub 16 is inserted and held, the catheter holding portion 56 being connected to the base portion 54. With this configuration, the holder 20 can be reliably fixed to the adhesive member 22 by the flat base portion 54. By inserting the distal portion of the catheter 30 (hub body 38) into the catheter holding portion 56, the distal portion of the hub body 38 can be held so as to be oriented toward the body surface 12a of the user 12.

The holder 20 has the port holding portion 58 that holds the side port 40 of the catheter hub 16, and the port holding portion 58 has the upper wall 66 that covers the side port 40 from above. Therefore, when the tube 18 connected to the side port 40 is pulled in a direction away from the body surface 12a (base portion 54), lifting of the side port 40 from the base portion 54 can be prevented by the port holding portion 58. Thus, even when the tube 18 is pulled, the catheter hub 16 can be prevented from being detached from the holder 20.

The base portion 54 has the notch 60a penetrating the base portion 54, and the lower part of the distal portion of the catheter hub 16 is placed in the notch 60a. Therefore, the distal end of the catheter 30 held by the catheter hub 16 can be located closer to the body surface 12a, and the dislodgement of the catheter 30 from the body can be effectively prevented.

The first embodiment includes: the holder 20 that holds the catheter hub 16; the adhesive member 22 that is fused to the back surface of the holder 20 and is to be affixed to the body surface 12a of the user 12; and the release sheet 72 that is overlaid on the adhesive surface 22a of the adhesive member 22 and is peeled off when the adhesive member 22 is fixed to the body surface 12a. The holder 20 and the adhesive member 22 are bonded to each other by the fusion portion 82. The fusion portion 82 includes the general fusion portion 84 and the peeling assisting portions 86a and 86b having a smaller bonding force than the general fusion portion 84. With this configuration, when the user 12 peels off the release sheet 72 from the adhesive surface 22a of the adhesive member 22, the peeling assisting portions 86a and 86b can prevent an excessive increase in the peel resistance of the release sheet 72. Therefore, the release sheet 72 can be easily peeled off from the adhesive member 22. It is possible to prevent the dislodgement of the catheter 30 that has already been left in place in the body when the release sheet 72 is peeled off.

In the fusion portion 82, the peeling assisting portions 86a and 86b are formed at positions where the maximum load (maximum peel resistance) is applied when the release sheet 72 is peeled off, so that the process of peeling off the release sheet 72 can be effectively assisted.

The peeling assisting portions 86a and 86b are formed as the non-fusion portions 88 provided by cutting a part of the general fusion portion 84. Thus, the peeling assisting portions 86a and 86b can be easily formed.

The peeling assisting portions 86a and 86b are located on the inner peripheral side of the fusion portion 82. Thus, an appropriate bonding force is obtained on the outer peripheral side of the fusion portion 82, and the adhesive member 22 can be fixed to the holder 20 with a desired bonding force. It is possible to prevent a rapid increase in the peel resistance of the release sheet 72 when the release sheet 72 is peeled off from the adhesive member 22.

The peeling assisting portions 86a and 86b are formed as fusion portions that are fused with a smaller bonding force than the bonding force of the general fusion portion 84. Thus, the peeling assisting portions 86a and 86b can be easily formed.

The adhesive member 22 includes: the first adhesive portion 68 through which the catheter 30 is inserted; and the second adhesive portion 70 to which the holder 20 is fixed, the second adhesive portion 70 being located at the proximal end of the first adhesive portion 68, and the peeling assisting portions 86a and 86b are provided between the holder 20 and the second adhesive portion 70. With this configuration, the holder 20 and the adhesive member 22 are fused to each other, whereby the release sheet 72 is less likely to be separated from the second adhesive portion 70. On the other hand, the peeling assisting portions 86a and 86b are provided between the holder 20 and the second adhesive portion 70, whereby the property of peeling off from the second adhesive portion 70 can be effectively increased.

The holder 20 has the port holding portion 58 that holds the side port 40 of the catheter hub 16, and the peeling assisting portions 86a and 86b are located at positions away from the port holding portion 58 in the circumferential direction of the fusion portion 82. This configuration can prevent a decrease in the bonding state between the adhesive member 22 and the holder 20 due to the peeling assisting portions 86a and 86b when a load is applied in the tensile direction to the tube 18 connected to the side port 40 and the side port 40 is pulled together with the tube 18.

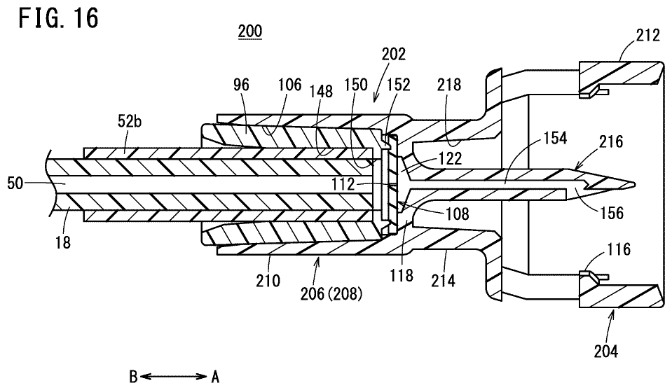

Next, a liquid medicine administration device 200 according to a second embodiment will be described with reference to FIGS. 16 to 18. Note that the same components as those of the liquid medicine administration device 10 according to the first embodiment described above are denoted by the same reference numerals, and the detailed description thereof will be omitted.

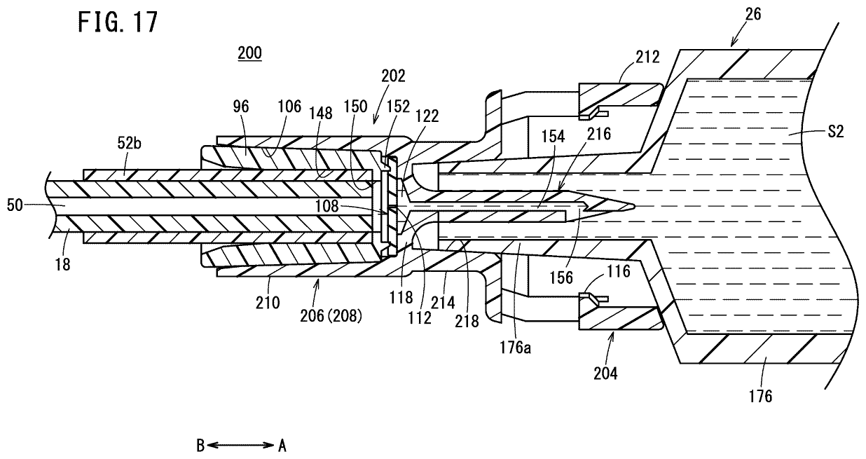

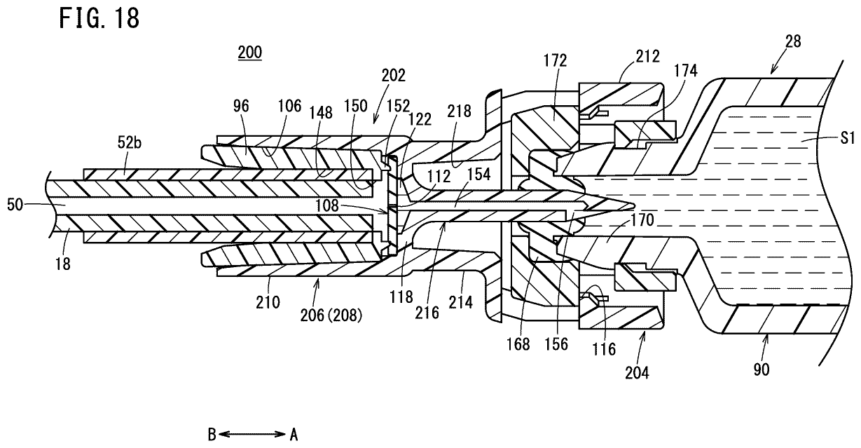

As illustrated in FIG. 16, a connector 202 of the liquid medicine administration device 200 according to the second embodiment includes a single connection member 208 including a first connection portion 204 connectable to a prefilled syringe 90 (see FIG. 18) of a device body 28 and a second connection portion 206 connectable to a priming solution delivery tool 26 (see FIG. 17) capable of delivering a priming solution S2. The first connection portion 204 includes connector tabs 116 and a hollow needle 216. A delivery nozzle portion 170 of the prefilled syringe 90 is to be connected to the first connection portion 204. The second connection portion 206 has a fitting hole 218. A distal nozzle portion 176a of the priming solution delivery tool 26 is to be connected to the second connection portion 206.

The connection member 208 is formed from a resin material, and includes a distal connection portion 210 formed at the distal end and holding the proximal end of a tube 18, a proximal connection portion 212 formed at the proximal end and to be connected to the device body 28, and an intermediate portion 214 to which the priming solution delivery tool 26 is to be connected, the intermediate portion 214 being located between the distal connection portion 210 and the proximal connection portion 212.

In the single connection member 208, the first connection portion 204 and the second connection portion 206 are different from each other. The proximal connection portion 212 serves as the first connection portion 204, and the intermediate portion 214 serves as the second connection portion 206.

A separation wall 118 of the intermediate portion 214 has a hollow needle 216 protruding in the proximal direction (direction of arrow A). The hollow needle 216 is formed integrally with the connection member 208.

The intermediate portion 214 has a fitting hole 218 into which the distal nozzle portion 176a of a barrel 176 is inserted. The fitting hole 218 faces the distal end of the hollow needle 216 and is formed in a tapered shape in the distal direction.

When the barrel 176 of the priming solution delivery tool 26 is connected from the proximal end of the connection member 208 illustrated in FIG. 17, the proximal end of the hollow needle 216 is inserted into the distal nozzle portion 176a of the barrel 176, and the distal nozzle portion 176a is fitted to the fitting hole 218 in a tapered manner. As a result, the priming solution S2 inside the barrel 176 is supplied in the distal direction through a needle hole 154 of the hollow needle 216, and the distal nozzle portion 176a of the barrel 176 and the fitting hole 218 of the connection member 208 are liquid-tightly connected.

When the prefilled syringe 90 of the device body 28 is connected from the proximal end of the connection member 208 illustrated in FIG. 18, the proximal end of the hollow needle 216 punctures the axial center of a packing 168, and the proximal end is inserted into the delivery nozzle portion 170 of the prefilled syringe 90. Thus, the liquid medicine S1 in the prefilled syringe 90 can be supplied in the distal direction through the needle hole 154 of the hollow needle 216.

In the liquid medicine administration device 200 according to the second embodiment, the first connection portion 204 connectable to the prefilled syringe 90 and the second connection portion 206 connectable to the priming solution delivery tool 26 are provided in the same connection member 208. Thus, the number of components and assembly man-hours can be reduced to decrease the manufacturing cost, and the connector 202 can be downsized in the axial direction.

The hollow needle 216 is formed integrally with the connection member 208, whereby the number of components and assembly man-hours of the liquid medicine administration device 200 can be reduced.

Note that the present invention is not limited to the above-described disclosure, and various configurations can be adopted without departing from the gist of the present invention.

1. A liquid medicine administration device comprising:
a catheter configured to be inserted into and left in place in a living body;
a tube configured to allow a liquid medicine to flow toward the catheter;
a connector located at a proximal portion of the tube, the connector being selectively connectable to (i) a priming solution delivery tool configured to deliver a priming solution from a distal portion of the priming solution delivery tool, and (ii) a prefilled syringe filled with the liquid medicine; and
a hollow needle located inside the connector and protruding in a proximal direction; wherein:
the connector comprises:
a first connection portion connectable to the prefilled syringe, and
a second connection portion located at a position different from the first connection portion and connectable to the priming solution delivery tool.

2. The liquid medicine administration device according to claim 1, wherein:
the first connection portion is located in a first connection member connected to the proximal portion of the tube, and
the second connection portion is located in a second connection member detachably attached to a proximal portion of the first connection member.

3. The liquid medicine administration device according to claim 2, wherein:
the second connection member comprises:
a first cylindrical body that is connectable to a distal portion of the priming solution delivery tool, and
a second cylindrical body that covers an outside of the first cylindrical body and is engaged with the first connection member.

4. The liquid medicine administration device according to claim 3, wherein:
the second cylindrical body comprises an arm that is located outside the first connection portion of the first connection member and is tiltable in a direction approaching or away from the first connection portion, and
the arm comprises an engagement portion that engages with an engagement hole formed in an outer peripheral surface of the first connection portion.

5. The liquid medicine administration device according to claim 1, wherein:
the first connection portion and the second connection portion are different portions of a single connection member.

6. The liquid medicine administration device according to claim 5, wherein:
the hollow needle is integrally molded with the connection member.

7. The liquid medicine administration device according to claim 5, wherein:
the second connection portion has a fitting hole into which a distal nozzle portion of the priming solution delivery tool is insertable and engageable, the fitting hole having a tapered shape and facing a distal end of the hollow needle.

8. The liquid medicine administration device according to claim 3, comprising:
a cap held by at least one of the first cylindrical body or the second cylindrical body, through which the hollow needle extends.

9. A connector for a liquid medicine administration device that comprises a catheter configured to be inserted into and left in place in a living body, and a tube configured to allow a liquid medicine to flow toward the catheter, and a hollow needle located inside the connector and protruding in a proximal direction, the connector being configured to be located at a proximal portion of the tube and comprising:
a first connection portion selectively connectable to a prefilled syringe filled with the liquid medicine, and
a second connection portion located at a position different from the first connection portion and selectively connectable to a priming solution delivery tool configured to deliver a priming solution from a distal portion of the priming solution delivery tool.

10. The connector according to claim 9, wherein:
the first connection portion is located in a first connection member configured to be connected to the proximal portion of the tube, and
the second connection portion is located in a second connection member detachably attached to a proximal portion of the first connection member.

11. The liquid medicine administration device according to claim 10, wherein:
the second connection member comprises:
a first cylindrical body that is connectable to a distal portion of the priming solution delivery tool, and
a second cylindrical body that covers an outside of the first cylindrical body and is engaged with the first connection member.

12. The connector device according to claim 11, wherein:
the second cylindrical body comprises an arm that is located outside the first connection portion of the first connection member and is tiltable in a direction approaching or away from the first connection portion, and
the arm comprises an engagement portion that engages with an engagement hole formed in an outer peripheral surface of the first connection portion.

13. The connector device according to claim 9, wherein:
the first connection portion and the second connection portion are different portions of a single connection member.

14. A connector assembly comprising:
the connector according to claim 13; and
the hollow needle integrally molded with the connection member.

15. The connector assembly according to claim 14, wherein:
the second connection portion has a fitting hole into which a distal nozzle portion of the priming solution delivery tool is insertable and engageable, the fitting hole having a tapered shape and facing a distal end of the hollow needle.

16. The connector assembly according to claim 14, comprising:
a cap held by at least one of the first cylindrical body or the second cylindrical body, through which the hollow needle extends.

17. A connector for a liquid medicine administration device, the connector being comprising:
a first connection member comprising a first connection portion selectively connectable to a prefilled syringe filled with the liquid medicine, and
a second connection member detachably attached to a proximal portion of the first connection member and comprising a second connection portion located at a position different from the first connection portion and selectively connectable to a priming solution delivery tool configured to deliver a priming solution from a distal portion of the priming solution delivery tool, wherein the second connection member comprises:
a first cylindrical body that is connectable to a distal portion of the priming solution delivery tool, and
a second cylindrical body that covers an outside of the first cylindrical body and is engaged with the first connection member.

18. The connector device according to claim 17, wherein:
the second cylindrical body comprises an arm that is located outside the first connection portion of the first connection member and is tiltable in a direction approaching or away from the first connection portion, and
the arm comprises an engagement portion that engages with an engagement hole formed in an outer peripheral surface of the first connection portion.

19. A connector assembly comprising:
the connector according to claim 17; and
a hollow needle integrally molded with the connection member.

20. The connector assembly according to claim 19, wherein:
the second connection portion has a fitting hole into which a distal nozzle portion of the priming solution delivery tool is insertable and engageable, the fitting hole having a tapered shape and facing a distal end of the hollow needle.
